(19) United States
(12) Patent Application Publication
(10) Pub. No.: US 2026/0321358 A1
(43) Pub. Date: Oct. 15, 2026

(54) RECORDING MEDIUM, INFORMATION PROCESSING SYSTEM, AND INFORMATION PROCESSING METHOD

(71) Applicant: Panasonic Holdings Corporation, Osaka (JP)

(72) Inventors: TAKAHIRO OOGOE, Osaka (JP); NOBUAKI NAGAO, Gifu (JP); MICHIO KATOUDA, Chiba (JP)

(21) Appl. No.: 19/686,860

(22) Filed: May 25, 2026

Related U.S. Application Data
(63) Continuation of application No. PCT/JP2024/043693, filed on Dec. 10, 2024.

(30) Foreign Application Priority Data
Dec. 13, 2023 (JP) 2023-210271
Nov. 8, 2024 (JP) 2024-196012

(51) Int. Cl. G16C 10/00

(52) U.S. Cl. CPC G16C 10/00

(57) ABSTRACT

A program causes a computer to perform steps of deriving two or more pieces of first state information indicative of at least one of energy or force on two or more potential energy surfaces among potential energy surfaces of a molecular structure including one or more molecules while using the molecular structure as input, and step of outputting second state information indicative of at least one of energy or force on a predetermined potential energy surface different from the two or more potential energy surfaces on the basis of the two or more pieces of first state information.

### BACKGROUND

### 1. Technical Field

The present disclosure relates to a technique of estimating at least one of energy and force of a molecular structure, and others.

### 2. Description of the Related Art

Japanese Unexamined Patent Application Publication No. 2022-027123 (hereinafter referred to as Patent Literature 1) discloses a technique of analyzing a chemical reaction from reactants to a product.

Simon Axelrod, Eugene Shakhnovich, Rafael Gomez-Bombarelli. “Thermal Half-Lives of Azobenzene Derivatives: Virtual Screening Based on Intersystem Crossing Using a Machine Learning Potential.” ACS Cent. Sci. 2023, 9, 166-176 (hereinafter referred to as Non Patent Literature 1) discloses research on virtual screening based on intersystem crossing using a machine learning potential.

### SUMMARY

One non-limiting and exemplary embodiment provides a program and others that make it possible to search for a reaction path that takes a reaction involving a transition from one potential energy surface to another potential energy surface into consideration in a reaction path search program using, as input, state information indicative of at least one of energy or force on one potential energy surface.

In one general aspect, the techniques disclosed here feature a program causing a computer to perform a process including: deriving two or more pieces of first state information indicative of at least one of energy or force on two or more potential energy surfaces among potential energy surfaces of a molecular structure including one or more molecules while using the molecular structure as input; and outputting second state information indicative of at least one of energy or force on a predetermined potential energy surface different from the two or more potential energy surfaces on the basis of the two or more pieces of first state information.

According to the present disclosure, it is possible to search for a reaction path that takes a reaction involving a transition from one potential energy surface to another potential energy surface into consideration even in a reaction path search program using, as input, state information indicative of at least one or energy or force on one potential energy surface.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a schematic view illustrating an example of a method for searching for a reaction path;

FIG. 2 is a flowchart illustrating an example of basic operation of a search program;

FIG. 3 is a schematic view illustrating an example of intersystem crossing;

FIG. 4A is an explanatory view for explaining a problem that occurs in a case where a search program to which only information on one potential energy surface can be input is used;

FIG. 4B illustrates an example of an external program that outputs energy or force on a potential energy surface corresponding to a reaction path that takes a reaction involving a transition from one potential energy surface to another potential energy surface into consideration;

FIG. 5 is a block diagram illustrating an overall configuration including an information processing system according to an embodiment;

FIG. 6 illustrates an example of output of second state information;

FIG. 7 is a flowchart illustrating an example of operation of the information processing system according to the embodiment;

FIG. 8 is an explanatory view for explaining ratios of periods required for a search process performed by a simulation device according to the embodiment;

FIG. 9 is an explanatory view for explaining accuracy of the search process performed by the simulation device according to the embodiment;

FIG. 10 is an explanatory view for explaining a problem of the technique disclosed in Non Patent Literature 1;

FIG. 11A illustrates an example of an input screen according to the embodiment; and

FIG. 11B illustrates an example of an output screen according to the embodiment.

### DETAILED DESCRIPTIONS

### Underlying Knowledge Forming Basis of the Present Disclosure

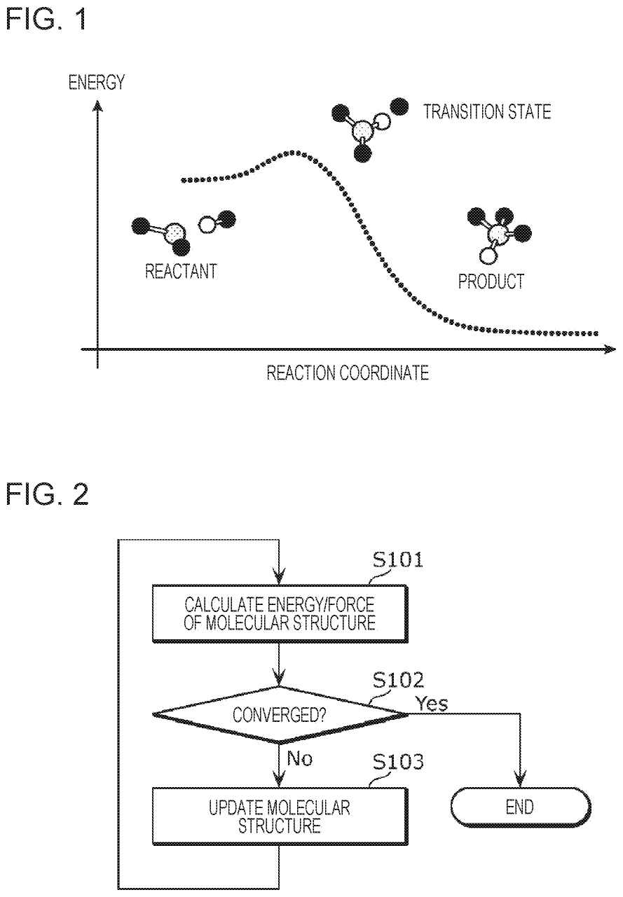

In recent years, for example, a search program based on a method such as Nudged Elastic Band (NEB) is used to analyze a chemical reaction of a molecular structure including one or more molecules. The NEB is a method for searching for a minimum energy path by optimizing a reaction path in a chemical reaction from a reactant to a product while using the reactant and the product as input. FIG. 1 is a schematic view illustrating an example of a reaction path search method.

FIG. 1 illustrates an example of a minimum energy path connecting a reactant, a transition state, and a product on a potential energy surface. The potential energy surface regards a reactant and a product as a single system (reaction system) and can be expressed by plotting potential energy of the reaction system against coordinates representing arrangement of all atoms included in a molecular structure. In FIG. 1, the vertical axis represents energy of the molecular structure, and the horizontal axis represents a reaction coordinate (a coordinate that is a one-dimensional representation of a reaction path connecting two stable structures on a potential energy surface). In FIG. 1, the transition state represents a molecular structure in a highest energy state in a process of a chemical reaction from the reactant to the product. By thus using a search program such as NEB, a user can intuitively examine a series of changes in molecular structure along a path of reaction coordinates (reaction path) and a change in energy of the molecular structure.

Basic processing performed by a search program is described with reference to FIG. 2. FIG. 2 is a flowchart illustrating an example of basic operation of the search program.

### Step S101

The search program performs processing for calculating energy and force of a molecular structure. In step S101, the search program calculates not only energy of the molecular structure, but also a gradient of the energy of the molecular structure, that is, force that acts on the molecular structure.

### Step S102

The search program performs processing for determining whether or not the energy and the force satisfy a convergence criterion on the basis of the energy and the force of the molecular structure calculated in step S101. In a case where it is determined that the energy and the force satisfy the convergence criterion (step S102: Yes), the search program ends the reaction path search processing. On the other hand, in a case where it is determined that the energy and the force do not satisfy the convergence criterion (step S102: No), the search program performs step S103.

### Step S103

The search program performs processing for updating the molecular structure (step S103). In step S103, the search program updates the molecular structure by adjusting positions of one or more atoms among all atoms included in the molecular structure so that force that acts on the one or more atoms is adjusted. Thereafter, the search program repeats steps S101 to S103 until it is determined that the energy and the force satisfy the convergence criterion.

Intersystem crossing sometimes occurs on a reaction path. The intersystem crossing is a radiationless transition between quantum states having different spin multiplicities. For example, in a case where a singlet state is nonradiatively converted to a triplet state or in a case where a triplet state is converted to a singlet state, this process is called intersystem crossing. In other words, the intersystem crossing is inversion of spin of an excited electron.

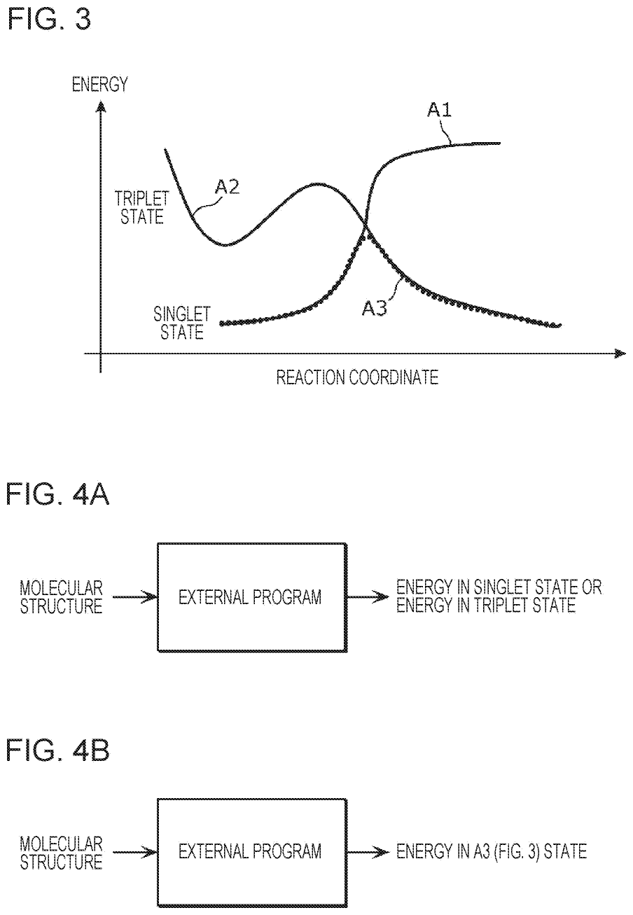

FIG. 3 is a schematic view illustrating an example of intersystem crossing. In FIG. 3, the vertical axis represents energy of a molecular structure, and the horizontal axis represents a reaction coordinate. In FIG. 3, the solid line A1 represents energy of the molecular structure in a singlet state, the solid line A2 represents energy of the molecular structure in a triplet state, and the dotted line A3 represents a reaction path to be searched for. In the example illustrated in FIG. 3, the reaction path to be searched for is a path passing the lower of the energy in the singlet state and the energy in the triplet state and is a path that takes intersystem crossing that occurs at an intersection of the solid line A1 and the solid line A2 into consideration.

As described above, in a case where a chemical reaction of a molecular structure is analyzed, it is important to be able to search for a reaction that takes intersystem crossing into consideration, in other words, a reaction involving a transition from one potential energy surface to another potential energy surface.

The search program can perform calculation of the energy in the singlet state and calculation of the energy in the triplet state by using an external program such as a quantum chemical calculation program (e.g., Gaussian (Registered Trademark)). However, the following problem occurs in a case where only information on one potential energy surface output by an external program can be input to the search program.

FIG. 4A is an explanatory view for explaining the problem that occurs in a case where only information on one potential energy surface can be input to the search program. In the search program, an external program such as a quantum chemical calculation program (e.g., Gaussian (Registered Trademark)) can be executed. However, in a case where the external program is used, the search program can perform processing using a molecular structure as input only once every time the molecular structure is updated. Accordingly, as illustrated in FIG. 4A, the external program can output only either energy in a singlet state or energy in a triplet state as energy of the input molecular structure.

That is, in a case where a search program to which only information on one potential energy surface can be input is used, both energy in a singlet state and energy in a triplet state cannot be calculated, and it is impossible to search for a reaction path that takes a reaction involving a transition from one potential energy surface to another potential energy surface into consideration.

In order to solve the above problem, a program according to a first aspect of the present disclosure causes a computer to perform a process including deriving two or more pieces of first state information indicative of at least one of energy or force on two or more potential energy surfaces among potential energy surfaces of a molecular structure including one or more molecules while using the molecular structure as input; and outputting second state information indicative of at least one of energy or force on a predetermined potential energy surface different from the two or more potential energy surfaces on the basis of the two or more pieces of first state information.

According to this aspect, it is possible to use an external program that outputs energy or force on a potential energy surface corresponding to a reaction path that takes a reaction involving a transition from one potential energy surface to another potential energy surface into consideration such as the one illustrated in FIG. 4B, and therefore it is possible to search for such a reaction path.

For example, in the first aspect, the program according to a second aspect of the present disclosure may be arranged such that the deriving includes outputting the two or more pieces of first state information by using not quantum chemical calculation, but a trained model that has been trained by machine learning while using the molecular structure as input.

According to this aspect, it is possible to use a trained model to search for a reaction involving a transition from one potential energy surface to another potential energy surface, and it is therefore easy to speed up a search for a reaction path that takes a reaction involving a transition from one potential energy surface to another potential energy surface into consideration.

In order to solve the above problem, an information processing system according to a third aspect of the present disclosure includes an acquirer that acquires molecular structure information indicative of a molecular structure including one or more molecules; a searcher that searches for a reaction path of the molecular structure while using the acquired molecular structure information as input; and an outputter that outputs, as a processing result of the searcher, second state information indicative of at least one of energy or force on a predetermined potential energy surface of the molecular structure information acquired by the acquirer, in which the searcher includes one or more inferencers that output two or more pieces of first state information indicative of at least one of energy or force on two or more potential energy surfaces among potential energy surfaces of a molecular structure while using the molecular structure as input, and the second state information indicates at least one of energy or force on the predetermined potential energy surface different from the two or more pieces of first state information output by the one or more inferencers. Note that the one or more inferencers may output the two or more pieces of first state information by using quantum chemical calculation.

According to this aspect, it is possible to search for a reaction path that takes a reaction involving a transition from one potential energy surface to another potential energy surface into consideration.

For example, in the information processing system according to a fourth aspect of the present disclosure, each of the one or more inferencers is a trained model that has been trained by machine learning, and the trained model has been trained to output the two or more pieces of first state information while using the molecular structure as input.

According to this aspect, it is possible to use a trained model to search for a reaction involving a transition from one potential energy surface to another potential energy surface, and it is therefore easy to speed up a search for a reaction path that takes a reaction involving a transition from one potential energy surface to another potential energy surface into consideration.

For example, in the third aspect, the information processing system according to a fifth aspect of the present disclosure may be arranged such that the outputter further outputs intersection information including at least one of energy at an intersection of the two or more potential energy surfaces, force at the intersection, or a molecular structure at the intersection.

According to this aspect, it is possible to obtain information concerning a molecular structure at an intersection of potential energy surfaces.

For example, in the fifth aspect, the information processing system according to a sixth aspect of the present disclosure may be arranged such that in a case where the second state information and the intersection information each include a molecular structure, a molecular structure at an extreme value of the energy indicated by the second state information matches a molecular structure indicated by the intersection information.

According to this aspect, it is possible to examine a molecular structure at an intersection of potential energy surfaces by examining an extreme value of energy or force indicated by the second state information.

For example, in any one of the third to sixth aspects, the information processing system according to a seventh aspect of the present disclosure may be arranged such that the predetermined potential energy surface is a surface that is differentiable at least once.

According to this aspect, energy indicated by the second state information smoothly changes also at an intersection of potential energy surfaces, and it is therefore possible to search for a reaction involving crossing by a conventional molecular structure updating method based on an energy derivative value.

For example, in any one of the third to sixth aspects, the information processing system according to an eighth aspect of the present disclosure may be arranged such that the two or more potential energy surfaces are surfaces that use different spin multiplicities as parameters.

According to this aspect, it is possible to search for a reaction involving a transition from one potential energy surface to another potential energy surface having different spin multiplicity, and it is therefore easy to search for a reaction that takes intersystem crossing into consideration.

For example, in the third or fourth aspect, the information processing system according to a ninth aspect of the present disclosure may be arranged such that the molecular structure is a molecular structure that exhibits intersystem crossing, and the one or more inferencers include a first inferencer that outputs first state information indicative of at least one of energy or force on a potential energy surface representing a singlet state of the molecular structure among the potential energy surfaces and a second inferencer that outputs first state information indicative of at least one of energy or force on a potential energy surface representing a triplet state of the molecular structure among the potential energy surfaces.

According to this aspect, it is possible to search for a reaction path that takes a reaction involving a transition from a potential energy surface representing a singlet state of a molecular structure to a potential energy surface representing a triplet state into consideration. Similarly, it is possible to search for a reaction path that takes a reaction involving a transition from a potential energy surface representing a triplet state of a molecular structure to a potential energy surface representing a singlet state into consideration.

For example, in the ninth aspect, the information processing system according to a tenth aspect of the present disclosure may be arranged such that the second state information output by the outputter indicates the lower energy of the energy on the potential energy surface representing the singlet state and the energy on the potential energy surface representing the triplet state.

According to this aspect, it is easy to derive an optimum reaction path by searching for a reaction path that takes a reaction involving a transition from a potential energy surface representing a singlet state of a molecular structure to a potential energy surface representing a triplet state into consideration. Similarly, it is easy to derive an optimum reaction path by searching for a reaction path that takes a reaction involving a transition from a potential energy surface representing a triplet state of a molecular structure to a potential energy surface representing a singlet state into consideration.

For example, in the ninth or tenth aspect, the information processing system according to an eleventh aspect of the present disclosure may be arranged such that the second state information output by the outputter indicates the force on a potential energy surface with lower energy between the potential energy surface representing the singlet state and the potential energy surface representing the triplet state.

According to this aspect, it is easy to derive an optimum reaction path by searching for a reaction path that takes a reaction involving a transition from a potential energy surface representing a singlet state of a molecular structure to a potential energy surface representing a triplet state into consideration. Similarly, it is easy to derive an optimum reaction path by searching for a reaction path that takes a reaction involving a transition from a potential energy surface representing a triplet state of a molecular structure to a potential energy surface representing a singlet state into consideration.

For example, in the fifth aspect, the information processing system according to a twelfth aspect of the present disclosure may be arranged such that the searcher further includes a processor that performs processing at the intersection of the two or more pieces of first state information output by the one or more inferencers and generates the second state information by combining regions of potential energy surfaces where at least one of energy or force is lower than at least one of energy or force at the intersection in the two or more pieces of first state information.

According to this aspect, it is easy to derive an optimum reaction path since regions of potential energy surfaces lower than at least one of energy or force at the intersection in the two or more pieces of first state information are combined.

An information processing method according to a thirteenth aspect of the present disclosure includes causing a computer to derive two or more pieces of first state information indicative of at least one of energy or force on two or more potential energy surfaces among potential energy surfaces of a molecular structure including one or more molecules while using the molecular structure as input; and causing the computer to output second state information indicative of at least one of energy or force on a predetermined potential energy surface different from the two or more potential energy surfaces on the basis of the two or more pieces of first state information.

According to this aspect, it is possible to use an external program that outputs energy or force on a potential energy surface corresponding to a reaction path that takes a reaction involving a transition from one potential energy surface to another potential energy surface into consideration such as the one illustrated in FIG. 4B, and therefore it is possible to search for such a reaction path.

Needless to say, the program, that is, the computer program of the present disclosure can be distributed via a computer-readable non-transitory recording medium such as a CD-ROM or over a communication network such as the Internet.

An embodiment is specifically described below with reference to the drawings.

The embodiment described below illustrates a general or specific example of the present disclosure. Numerical values, shapes, materials, constituent elements, the way in which the constituent elements are disposed and connected, steps, the order of steps, and the like in the embodiment below are examples and do not limit the present disclosure. Among constituent elements in the embodiment below, constituent elements that are not described in independent claims indicating highest concepts are described as optional constituent elements. Each drawing is a schematic view and is not necessarily strict illustration. In the drawings, identical constituent elements are given identical reference signs.

An information processing system according to the embodiment of the present disclosure may be configured so that all constituent elements are included in a single computer or may be configured as a system in which constituent elements are distributed into computers.

### EMBODIMENT

An information processing system (an information processing method, or a program) according to the embodiment of the present disclosure is described with reference to the drawings.

### Configuration

First, a configuration of the information processing system used in the embodiment is described.

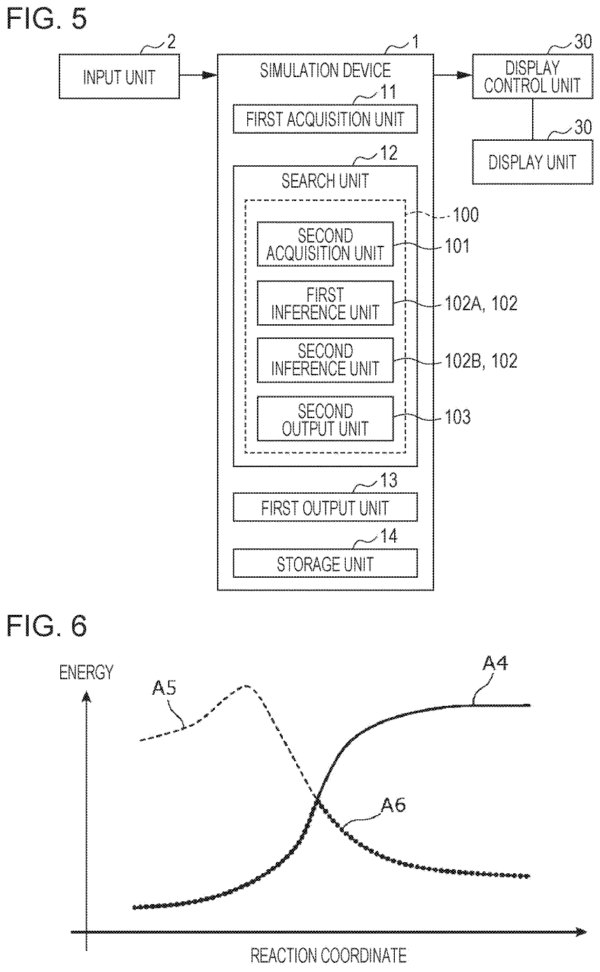

FIG. 5 is a block diagram illustrating an overall configuration including an information processing system 100 according to the embodiment. The information processing system 100 is, for example, realized by a simulation device 1 configured as computer such as a personal computer or a server. The information processing system 100 may be, for example, realized by edge computing or may be, for example, realized by cloud computing. In the embodiment, it is assumed that the simulation device 1 is a desktop computer.

The simulation device 1 includes a first acquisition unit 11, a search unit 12, a first output unit 13, and a storage unit 14. The simulation device 1 executes a search program for searching for a reaction path in a chemical reaction of a molecular structure by using the above search method such as NEB. Note that examples of the search program that can be executed by the simulation device 1 include a program using a metadynamics method, a program using a molecular dynamics method in addition to the program using NEB. In the embodiment, the simulation device 1 executes the search program using NEB.

An input unit 2, a display control unit 30, and a display unit 3 are connected to the simulation device 1. The input unit 2, the display control unit 30, and the display unit 3 are, for example, realized by an information terminal used by a user such as a smartphone, a tablet terminal, or a personal computer.

The input unit 2 and the display control unit 30 may be connected to the simulation device 1 over a network such as a Local Area Network (LAN) or may be connected to the simulation device 1 over a network such as the Internet.

The input unit 2 is an input interface that receives user's input and is, for example, realized by a keyboard, a touch sensor, a touch pad, a mouse, or the like. The input unit 2 receives a user's input operation and outputs a signal according to the input operation to the information processing system 100. Although the display unit 3 and the input unit 2 are independent of each other in the present disclosure, the display unit 3 and the input unit 2 may be integral with each other, for example, as a touch panel. Although the information processing system 100 does not include the display unit 3 and the input unit 2 in the present disclosure, the information processing system 100 may include the display unit 3 and the input unit 2.

The input unit 2 receives input of initial molecular structure information concerning an initial molecular structure including one or more molecules desired by a user. The information received by the input unit 2 is, for example, a composition formula indicative of a composition of a reactant for which the user wants to examine a process of a chemical reaction.

The display control unit 30 causes the display unit 3 to display an image or the like on the basis of information output from the first output unit 13 of the simulation device 1.

The display unit 3 displays an image or the like by being controlled by the display control unit 30. The display unit 3 is, for example, a liquid crystal display, a plasma display, an organic Electro-Luminescence (EL) display, or the like, but is not limited to this. Note that a device including the display unit 3 and the display control unit 30 may be called a display unit.

The first acquisition unit 11 acquires the initial molecular structure information received by the input unit 2.

The search unit 12 searches for a reaction path in a chemical reaction of the initial molecular structure indicated by the initial molecular structure information acquired by the first acquisition unit 11. In the embodiment, the search unit 12 searches for the reaction path in accordance with NEB. Specifically, the search unit 12 searches for the reaction path in accordance with the above-described example of basic operation of the search program using NEB illustrated in FIG. 2.

The first output unit 13 outputs an image or the like indicative of a processing result of the search unit 12 to the display control unit 30, and thus the image or the like is displayed on the display unit 3. For example, the first output unit 13 causes an image indicative of a reaction path including a reactant (corresponding to the initial molecular structure), a transition state, and a product such as the one illustrated in FIG. 1 to be displayed on the display unit 3.

The storage unit 14 is a recording medium for storing data (including a program) used in various kinds of processing that can be performed by the simulation device 1. The recording medium is, for example, a hard disk drive, a Random Access Memory (RAM), a Read Only Memory (ROM), a semiconductor memory, or the like. Note that such a recording medium may be volatile or may be nonvolatile.

In the embodiment, the search unit 12 includes a second acquisition unit 101, one or more inference units 102, and a second output unit 103. The second acquisition unit 101, the one or more inference units 102, and the second output unit 103 are constituent elements of the information processing system 100. The second acquisition unit 101 corresponds to an “acquisition unit” in the information processing system 100, and the second output unit 103 corresponds to an “output unit” in the information processing system 100. Note that the second acquisition unit 101 need not necessarily be included in the constituent elements of the information processing system 100.

The second acquisition unit 101 acquires molecular structure information concerning a molecular structure including one or more molecules. The second acquisition unit 101 acquires, as the molecular structure information, the initial molecular structure information acquired by the first acquisition unit 11 at a time of start of a search for the reaction path by the search unit 12. Thereafter, the second acquisition unit 101 acquires molecular structure information concerning the updated molecular structure every time the molecular structure is updated by the search unit 12.

The one or more inference units 102 use a molecular structure as input and output two or more pieces of first state information by using, for example, a trained model trained by quantum chemical calculation or machine learning. The two or more pieces of first state information indicate at least one of energy or force on two or more potential energy surfaces among potential energy surfaces of the molecular structure indicated by the molecular structure information acquired by the second acquisition unit 101, respectively. The one or more inference units 102 are units that perform an inference step in the information processing method of the present disclosure.

In the embodiment, the one or more inference units 102 include a first inference unit 102A and a second inference unit 102B.

The first inference unit 102A outputs first state information indicative of energy and force on a first potential energy surface corresponding to the molecular structure indicated by the molecular structure information acquired by the second acquisition unit 101 by using quantum chemical calculation or a first trained model. The first potential energy surface is a potential energy surface of the molecular structure in a singlet state, that is, a potential energy surface for spin multiplicity “1”. Note that the energy and the force on the first potential energy surface are values obtained by substituting a value of the molecular structure indicated by the horizontal axis of the graph of FIG. 6, which will be described later, that is, a value of a reaction coordinate of the molecular structure into a function of the first potential energy surface. Note that the molecular structure is a structure indicated by the molecular structure information acquired by the second acquisition unit 101.

The second inference unit 102B outputs first state information indicative of energy and force on a second potential energy surface corresponding to the molecular structure indicated by the molecular structure information acquired by the second acquisition unit 101 by using quantum chemical calculation or a second trained model. The second potential energy surface is a potential energy surface of the molecular structure in a triplet state, that is, a potential energy surface for spin multiplicity “3”. Note that the energy and the force on the second potential energy surface are values obtained by substituting a value of the molecular structure indicated by the horizontal axis of the graph of FIG. 6, which will be described later, that is, a value of a reaction coordinate of the molecular structure into a function of the first potential energy surface. Note that the molecular structure is a structure indicated by the molecular structure information acquired by the second acquisition unit 101.

As described above, in the embodiment, the two or more potential energy surfaces (the first potential energy surface and the second potential energy surface in this example) are surfaces using different spin multiplicities as parameters.

In the embodiment, the trained model is a machine learning potential model, and is, for example, a graph neural network that uses a graph structure as input. The graph neural network is, for example, Crystal Graph Convolutional Neural Network (CGCNN), Material Graph Network (MEGNet), or the like. Note that the trained model is not limited to a graph neural network and may be, for example, a method using a symmetry function.

The following describes a process of machine learning of the trained model. In particular, the following describes a process of machine learning of the first trained model and the second trained model. In the following description, the first trained model before completion of machine learning is referred to as a “first model”, and the second trained model before completion of machine learning is referred to as a “second model”.

First, a process of machine learning of the first trained model is described. In the process, the first model is trained by machine learning to output, in response to input of a molecular structure, a value (hereinafter also referred to as a “first state value”) indicative of at least one of energy or force on the first potential energy surface of the molecular structure by using a first training dataset. The first training dataset includes a molecular structure as input data and the first state value corresponding to the molecular structure as truth data.

First, a device that performs machine learning (hereinafter referred to as a “training device”) converts coordinates and kinds of atoms of the molecular structure included in the first training dataset into a graph structure. In the graph structure, nodes correspond to the atoms of the molecular structure, and edges correspond to coupling between atoms of the molecular structure. Next, the training device inputs the graph structure thus obtained to the first model. Then, the training device compares a predicted value of the first state value output from the first model and a first state value as truth data. In a case where the predicted value of the first state value is deviated from the first state value as truth data, the training device updates a weight of the first model. In this way, the training device trains the first model by supervised learning using a large number of first training datasets.

Next, a process of machine learning of the second trained model is described. In the process, the second model is trained by machine learning to output, in response to input of a molecular structure, a value (hereinafter also referred to as a “second state value”) indicative of at least one of energy or force on the second potential energy surface of the molecular structure by using a second training dataset. The second training dataset includes a molecular structure as input data and the second state value corresponding to the molecular structure as truth data.

First, the training device converts coordinates and kinds of atoms of the molecular structure included in the second training dataset into a graph structure. Next, the training device inputs the graph structure thus obtained to the second model. Then, the training device compares a predicted value of the second state value output from the second model and a second state value as truth data. In a case where the predicted value of the second state value is deviated from the second state value as truth data, the training device updates a weight of the second model. In this way, the training device trains the second model by supervised learning using a large number of second training datasets. The above describes a process of machine learning of the trained model.

The second output unit 103 outputs second state information on the basis of the two or more pieces of first state information output by the one or more inference units 102. The second state information indicates at least one of energy or force on a predetermined potential energy surface different from the two or more potential energy surfaces. The second output unit 103 is a unit that performs an output step in the information processing method according to the present disclosure. The second state information output by the second output unit 103 is referred to in a process for determining whether or not energy has converged and a process for updating a molecular structure performed by the search unit 12 of the simulation device 1.

In the embodiment, the second output unit 103 outputs the second state information indicative of both of energy and force on a third potential energy surface on the basis of the first state information output by the first inference unit 102A and the first state information output by the second inference unit 102B. The third potential energy surface is a surface different from the first potential energy surface and the second potential energy surface and is a surface that satisfies a predetermined condition.

The predetermined condition can include, for example, a condition that the lower of the energy on the first potential energy surface and the energy on the second potential energy surface is employed. Note that the predetermined condition is not limited to the above condition and may be another condition.

FIG. 6 illustrates an example of output of the second state information. FIG. 6 illustrates an example of output of the second state information in a case where the above condition is satisfied as the predetermined condition. In FIG. 6, the vertical axis represents energy of a molecular structure, and the horizontal axis represents a reaction coordinate. In FIG. 6, the solid line A4 represents energy on the first potential energy surface, that is, energy of the molecular structure in a singlet state. In FIG. 6, the broken line A5 represents energy on the second potential energy surface, that is, energy of the molecular structure in a triplet state. In FIG. 6, the dotted line A6 represents energy on the third potential energy surface that satisfies the above condition, that is, the lower of the energy in the singlet state and the energy in the triplet state.

In the example illustrated in FIG. 6, a path (reaction path) of the reaction coordinate is a path that takes intersystem crossing whose energy is local maximum at an intersection of the solid line A4 and the broken line A5 into consideration.

As described above, it can be said that the second output unit 103 outputs intersection information including energy at an intersection at which two or more potential energy surfaces (the first potential energy surface and the second potential energy surface in this example) intersect. Note that the intersection information may be information including at least one of energy at the intersection, force at the intersection, or a molecular structure at the intersection.

As described above, in a case where the second state information and the intersection information each include a molecular structure, the molecular structure at an extreme value of energy indicated by the second state information (i.e., a local minimum value or a local maximum value of energy on the third potential energy surface) matches the molecular structure indicated by the intersection information.

In the embodiment, the predetermined potential energy surface (i.e., the third potential energy surface) is a surface that is differentiable at least once and is a smooth surface that does not include a steep convex shape. Since energy smoothly changes at an intersection, that is, a position where intersystem crossing occurs on the path (reaction path) of the reaction coordinate, it is possible to search for a reaction involving crossing by a conventional molecular structure updating method based on an energy derivative value.

### Operation

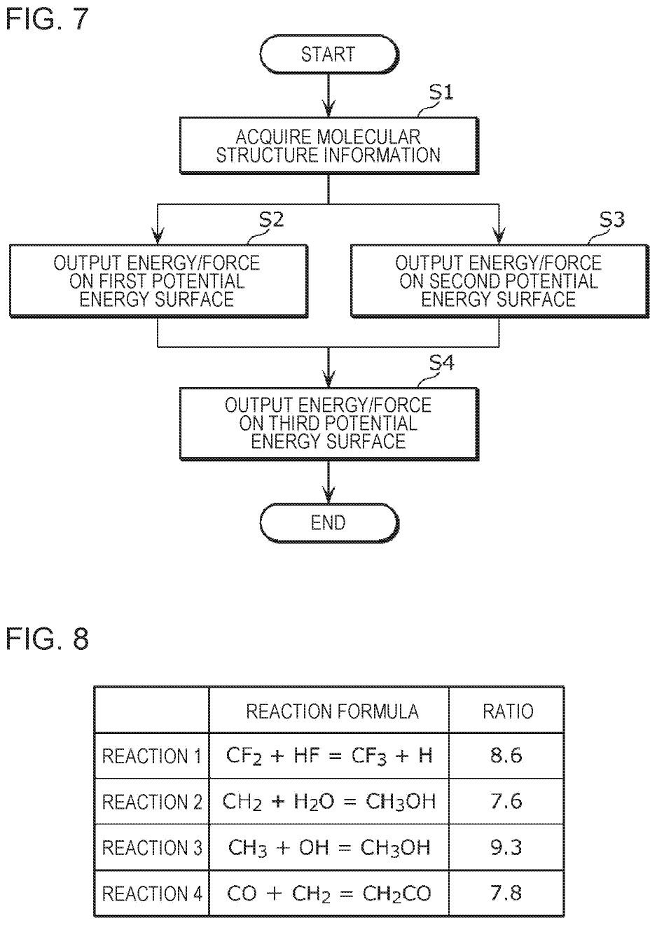

Operation (i.e., an information processing method) of the information processing system 100 according to the embodiment is described below. FIG. 7 is a flowchart illustrating an example of operation of the information processing system 100 according to the embodiment. In the example illustrated in FIG. 7, it is assumed that the one or more inference units 102 include the first inference unit 102A and the second inference unit 102B. Note that step S1 described below need not necessarily be included in the operation (information processing method) of the information processing system 100.

### Step S1

The second acquisition unit 101 acquires molecular structure information. In this step, the second acquisition unit 101 acquires the initial molecular structure information acquired by the first acquisition unit 11 of the simulation device 1 as the molecular structure information at a time of start of a search for a reaction path by the search unit 12 of the simulation device 1. Thereafter, every time the molecular structure is updated by the search unit 12, the second acquisition unit 101 acquires molecular structure information concerning the updated molecular structure.

### Step S2

The first inference unit 102A outputs first state information indicative of energy and force on a first potential energy surface corresponding to a molecular structure indicated by the molecular structure information acquired by the second acquisition unit 101 by using the first trained model.

### Step S3

The second inference unit 102B outputs first state information indicative of energy and force on a second potential energy surface corresponding to the molecular structure indicated by the molecular structure information acquired by the second acquisition unit 101 by using the second trained model.

Although the information processing system 100 perform step S2 and step S3 in parallel in the embodiment, step S3 may be performed after step S2 or step S2 may be performed after step S3.

### Step S4

The second output unit 103 outputs second state information indicative of both energy and force on a third potential energy surface on the basis of the first state information output by the first inference unit 102A and the first state information output by the second inference unit 102B.

The information processing system 100 repeatedly performs steps S1 to S4 every time the molecular structure is updated by the search unit 12 until the search process performed by the search unit 12 of the simulation device 1 ends.

### Advantages

As described above, the program used for the information processing system 100 according to the embodiment causes a computer to perform a derivation step of deriving two or more pieces of first state information indicative of at least one of energy or force on two or more potential energy surfaces among potential energy surfaces of a molecular structure including one or more molecules while using the molecular structure as input and an output step of outputting second state information indicative of at least one of energy or force on a predetermined potential energy surface different from the two or more potential energy surfaces on the basis of the two or more pieces of first state information.

Note that the computer is, for example, the information processing system 100. The derivation step corresponds to steps S2 and S3 in FIG. 7, and the output step corresponds to step S4 in FIG. 7. Although the first trained model and the second trained model are used in the embodiment, these trained models need not necessarily be used.

This makes it possible to search for a reaction path that takes a reaction involving a transition from one potential energy surface to another potential energy surface into consideration. The information processing method performed by such a program can also produce an effect similar to that produced by the above program.

The derivation step includes outputting two or more pieces of first state information by using a trained model trained by machine learning while using the one molecular structure as input.

This makes it possible to use a trained model to search for a reaction involving a transition from one potential energy surface to another potential energy surface, thereby achieving an advantage of speeding up search for a reaction path that takes a reaction involving a transition from one potential energy surface to another potential energy surface into consideration.

The information processing system 100 according to the embodiment includes the second acquisition unit 101, the search unit 12 including the one or more inference units 102, and the second output unit 103. Note that although the search unit 12 includes the second acquisition unit 101 and the second output unit 103 in the example of FIG. 5, the search unit 12 need not necessarily include these units. The second acquisition unit 101 acquires molecular structure information indicative of a molecular structure including one or more molecules. The search unit 12 uses the acquired molecular structure information as input and searches for a reaction path of the molecular structure. The second output unit 103 outputs, as a processing result of the search unit 12, second state information indicative of at least one of energy or force on a predetermined potential energy surface of the molecular structure information acquired by the second acquisition unit 101. The search unit 12 includes the one or more inference units 102 that use a molecular structure as input and output two or more pieces of first state information indicative of at least one of energy or force on two or more potential energy surfaces among potential energy surfaces of the molecular structure. The second state information indicates at least one of energy or force on a predetermined potential energy surface different from the two or more pieces of first state information output by the one or more inference units 102.

This produces an advantage that it is possible to search for a reaction path that takes a reaction involving a transition from one potential energy surface to another potential energy surface into consideration.

For example, in the information processing system 100 according to the embodiment, each of the one or more inference units 102 is a trained model that is trained by machine learning, and the trained model is trained to output two or more pieces of first state information while using a molecular structure as input.

This makes it possible to use a trained model to search for a reaction involving a transition from one potential energy surface to another potential energy surface, thereby producing an advantage that it is easy to speed up a search for a reaction path that takes a reaction involving a transition from one potential energy surface to another potential energy surface into consideration.

Specifically, it can be said that the search unit 12 includes a processing unit that performs processing at an intersection at which the two or more pieces of first state information output by the one or more inference units 102 intersect and generates second state information by combining regions of potential energy surfaces where at least one of energy or force is lower than at least one of energy or force at the intersection in the two or more pieces of first state information.

This produces an advantage that it is easy to derive an optimal reaction path since regions of potential energy surfaces where at least one of energy or force is lower than at least one of energy or force at the intersection in the two or more pieces of first state information are combined.

That is, the information processing system 100 according to the embodiment can realize the process performed by the one or more inference units 102 and the process performed by the second output unit 103 as one process of outputting at least one of energy or force while using a molecular structure as input. Therefore, in a case where the processing performed by the information processing system 100 according to the embodiment is employed as an external program in a search program using NEB or the like, the external program can calculate both of energy in a singlet state and energy in a triplet state and output lower energy.

Therefore, the information processing system 100 according to the embodiment has an advantage that it is easy to speed up a search for a reaction path that takes a reaction involving a transition from one potential energy surface to another potential energy surface into consideration since it is possible to use a trained model to search for a reaction involving a transition from one potential energy surface to another potential energy surface.

Specific examples of the advantages of the information processing system 100 according to the embodiment are described with reference to the drawings.

FIG. 8 is an explanatory view for explaining ratios of periods required for the search process performed by the simulation device 1 according to the embodiment. FIG. 8 shows results of the search process performed on four chemical reactions, specifically, “REACTION 1”, “REACTION 2”, “REACTION 3”, and “REACTION 4”. In FIG. 8, the “REACTION FORMULA” shows reactants and a product in each of the chemical reactions. In FIG. 8, the “RATIO” shows how many times longer the NEB-based search process using a quantum chemical calculation program takes as compared to the NEB-based search process using the machine learning potential model according to the embodiment.

As illustrated in FIG. 8, in each of the chemical reactions, a period required for the NEB-based search process using the machine learning potential model according to the embodiment is markedly shortened as compared with a period required for the NEB-based search process using a quantum chemical calculation program. That is, according to the information processing system 100 according to the embodiment, it is easy to speed up a search process using a search program.

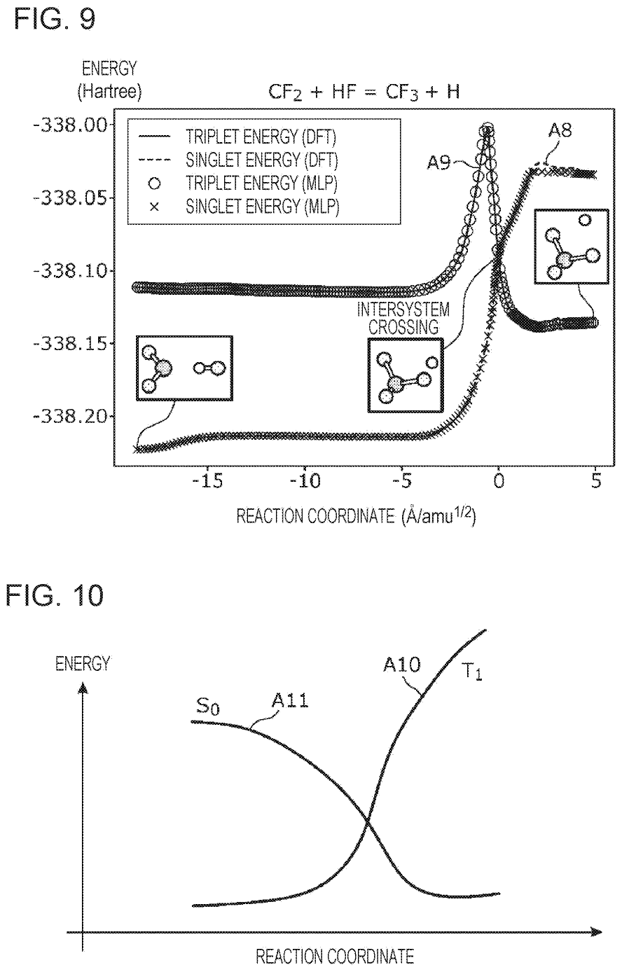

FIG. 9 is an explanatory view for explaining accuracy of the search process performed by the simulation device 1 according to the embodiment. FIG. 9 shows a calculation result of energy of a molecular structure in a case where the search process is performed on the chemical reaction of “REACTION 1”. In FIG. 9, the vertical axis represents energy, and the horizontal axis represents a reaction coordinate. In FIG. 9, the broken line A8 indicates a calculation result of energy in a singlet state using a quantum chemical calculation program (Gaussian (registered trademark) in this example), and the solid line A9 indicates a calculation result of energy in a triplet state using the quantum chemical calculation program. In FIG. 9, the circle marks indicate a calculation result of energy in a triplet state using the machine learning potential model according to the embodiment, and the cross marks indicate a calculation result of energy in a singlet state using the machine learning potential model according to the embodiment.

As illustrated in FIG. 9, the calculation result of energy using the machine learning potential model according to the embodiment is almost identical to the calculation result of energy using the quantum chemical calculation program. That is, according to the information processing system 100 according to the embodiment, it is easy to calculate at least one of energy or force of a molecular structure with accuracy equivalent to that in a case where the quantum chemical calculation program is used.

Note that although the advantages of using the information processing system 100 according to the embodiment in a case where the search process is performed on a chemical reaction of a small-molecule system have been described in the above specific example, advantages similar to those described above can be produced by using the information processing system 100 according to the embodiment in a case where the search process is performed on a chemical reaction of a large-molecule system. In particular, in a case where the search process is performed on a chemical reaction of a large-molecule system, it is necessary to calculate energy of a large number of molecular structures as compared with a case where the search process is performed on a chemical reaction of a small-molecule system, and therefore it can be expected that a period required for the search process is more effectively shortened.

The following describes advantages over the technique disclosed in Patent Literature 1 and the technique disclosed in Non Patent Literature 1.

The technique disclosed in Patent Literature 1 performs a search for a reaction path using NEB, but does not perform a search for a reaction path that takes intersystem crossing into consideration.

The technique disclosed in Non Patent Literature 1 searches for intersystem crossing based on sign inversion of a difference between energy in a singlet state and energy in a triplet state after generating candidates for a transition state by a metadynamics method using a neural network. However, according to the technique disclosed in Non Patent Literature 1, the candidates for a transition state are searched for in consideration of only a spin state of a singlet state or a triplet state, and therefore the following problem occurs.

FIG. 10 is an explanatory view for explaining the problem of the technique disclosed in Non Patent Literature 1. In FIG. 10, the vertical axis represents energy of a molecular structure, and the horizontal axis represents a reaction coordinate. In FIG. 10, the solid line A10 represents energy in a singlet state, and the solid line A11 represents energy in a triplet state. Although intersystem crossing occurs in the example illustrated in FIG. 10, a transition state is present on neither a reaction path of the energy in the singlet state nor a reaction path of the energy in the triplet state. Therefore, the technique disclosed in Non Patent Literature 1 cannot generate candidates for a transition state and therefore cannot search for the intersystem crossing.

On the other hand, as has been already described above, the information processing system 100 according to the embodiment has an advantage that it is possible to search for a reaction path that takes intersystem crossing into consideration since an external program that outputs energy and force on an energy surface corresponding to intersystem crossing reaction is used. Furthermore, as has been already described above, the information processing system 100 according to the embodiment has an advantage that it is easy to speed up processing for calculating energy of a molecular structure and shorten a period required for a search for a reaction path since energy of a molecular structure is calculated by using a machine learning potential model (trained model).

### Example of Displayed Screen

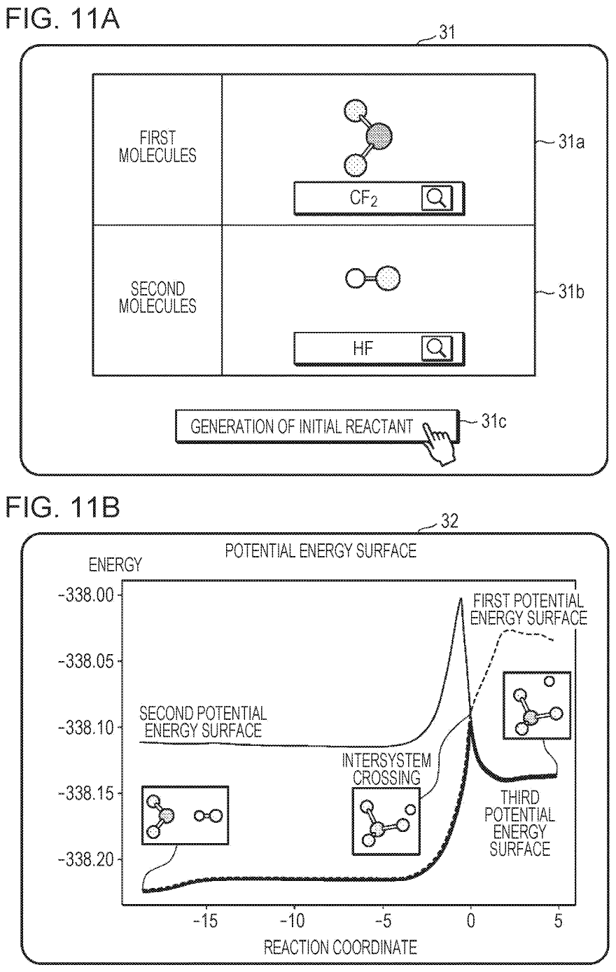

FIG. 11A illustrates an example of an input screen displayed on the display unit 3.

The display control unit 30 displays, for example, an input screen 31 illustrated in FIG. 11A on the display unit 3 to acquire the molecular structure information. The input screen 31 includes a first input field 31a, a second input field 31b, and a generation button 31c. In response to user's input operation on the input unit 2, the display control unit 30 displays first molecules designated by a user in the first input field 31a and displays second molecules designated by the user in the second input field 31b. The first molecules and the second molecules may be displayed as a composition formula or may be displayed as an image showing a molecular structure (i.e., an initial molecular structure). Note that the first molecules and the second molecules are initial reactants. In a case where the generation button 31c is selected in response to user's input operation on the input unit 2, the simulation device 1 causes the simulation device 1, that is, the information processing system 100 to perform the operation illustrated in FIG. 7.

FIG. 11B illustrates an example of an output screen displayed on the display unit 3.

The display control unit 30 displays, for example, an output screen 32 illustrated in FIG. 11B on the display unit 3 when the search process performed by the search unit 12 of the simulation device 1 ends. The output screen 32 shows a reaction path including a reactant (corresponding to an initial molecular structure), a transition state, and a product. The user can easily check a reaction path that takes a reaction involving a transition from a first potential energy surface to a second potential energy surface into consideration, that is, a third potential energy surface by seeing the output screen 32.

### Modifications

Although the program, the information processing system, the information processing method, and others according to one or more aspect of the present disclosure have been described above on the basis of the embodiment, the present disclosure is not limited to the above embodiment. Various modifications that may be conceived by those skilled in the art without departing from the spirit of the present disclosure may also be encompassed within the scope of the present disclosure.

For example, although the inference unit 102 outputs first state information indicative of at least one of energy or force on one of potential energy surfaces in the above embodiment, this is not restrictive. For example, the inference unit 102 may be configured to output two or more pieces of first state information indicative of at least one of energy or force on two or more potential energy surfaces.

Specifically, although the first inference unit 102A outputs the first state information indicative of energy and force on the first potential energy surface and the second inference unit 102B outputs the first state information indicative of energy and force on the second potential energy surface in the embodiment, a single inference unit 102 may be configured to output these two pieces of state information.

For example, although the one or more inference units 102 are two inference units 102 and output two pieces of first state information concerning two potential energy surfaces of a molecular structure in the above embodiment, this is not restrictive. For example, the one or more inference units 102 may be three or more inference units 102 and output three or more pieces of first state information concerning three or more potential energy surfaces of a molecular structure.

For example, although the two or more potential energy surfaces include a potential energy surface in a singlet state of a molecular structure and a potential energy surface in a triplet state of the molecular structure in the above embodiment, this is not restrictive. For example, the two or more potential energy surfaces may include a potential energy surface in a doublet state, a potential energy surface in a quartet state, a potential energy surface in a quintet state of a molecular structure, or the like.

For example, although the information processing system 100 is realized as a part of the search unit 12 of the simulation device 1 in the above embodiment, this is not restrictive. For example, the information processing system 100 may be realized by one or more devices different from the simulation device 1.

Note that in the above embodiment, each of the constituent elements may be configured as dedicated hardware or may be realized by execution of a software program suitable for the constituent element. Each of the constituent elements may be realized by a program execution unit such as a central processing unit (CPU) or a processor reading out and executing a software program recorded on a recording medium such as a hard disk or a semiconductor memory.

Note that the following cases are also encompassed within the present disclosure.
(1) The at least one device described above is specifically a computer system that includes a microprocessor, a Read Only Memory (ROM), a Random Access Memory (RAM), a hard disk unit, a display unit, a keyboard, a mouse, and the like. A computer program is stored in the RAM or the hard disk unit. The microprocessor operates in accordance with the computer program, and thus the at least one device accomplishes a function thereof. The computer program is a combination of command codes indicating a command given to a computer for accomplishment of a predetermined function.
(2) Part of or all of constituent elements that constitute the at least one device may include a single system large scale integration (LSI). The system LSI is a super-multi-function LSI produced by integrating constituents on a single chip and is specifically a computer system including a microprocessor, a ROM, a RAM, and the like. A computer program is stored in the RAM. The microprocessor operates in accordance with the computer program, and thus the system LSI accomplishes a function thereof.
(3) Part of or all of constituent elements that constitute the at least one device may include an IC card that can be detachably attached to the device or a stand-alone module. The IC card or the module is a computer system that includes a microprocessor, a ROM, a RAM, and the like. The IC card or the module may include the super-multi-function LSI. The microprocessor operates in accordance with a computer program, and thus the IC card or the module accomplishes a function thereof. The IC card or the module may have tamper resistance.
(4) The present disclosure may be the method described above. The present disclosure may be a computer program for causing a computer to realize the method or may be a digital signal embodying the computer program.

The present disclosure may be a computer-readable recording medium, such as a flexible disc, a hard disk, a Compact Disc-ROM (CD-ROM), a DVD, a DVD-ROM, a DVD-RAM, a BD (Blu-ray (Registered Trademark) Disc), or a semiconductor memory on which the computer program or the digital signal is recorded. The present disclosure may be the digital signal recorded on such a recording medium.

The present disclosure may be the computer program or the digital signal transmitted over an electric communication line, a wireless or wired communication line, a network represented by the Internet, data broadcasting, or the like.

The program or the digital signal may be executed by another independent computer system by transporting the program or the digital signal on the recording medium or transporting the program or the digital signal over the network or the like.

For example, the present disclosure produces an effect that user's prediction of a chemical reaction can be appropriately assisted and can be used for a computer device or a system for displaying information concerning a prediction process.

What is claimed is:

1. A nonvolatile computer-readable recording medium storing a program causing a computer to perform a process comprising:
deriving two or more pieces of first state information indicative of at least one of energy or force on two or more potential energy surfaces among potential energy surfaces of a molecular structure including one or more molecules while using the molecular structure as input; and
outputting second state information indicative of at least one of energy or force on a potential energy surface including an intersection of the two or more potential energy surfaces on a basis of the two or more pieces of first state information.

2. The nonvolatile computer-readable recording medium according to claim 1, wherein
the deriving includes outputting the two or more pieces of first state information by using a trained model that has been trained by machine learning while using the molecular structure as input.

3. An information processing system comprising:
an acquirer that acquires molecular structure information indicative of a molecular structure including one or more molecules;
a searcher that searches for a reaction path of the molecular structure while using the acquired molecular structure information as input; and
an outputter that outputs, as a processing result of the searcher, second state information indicative of at least one of energy or force on a predetermined potential energy surface of the molecular structure information acquired by the acquirer, wherein
the searcher includes one or more inferencers that output two or more pieces of first state information indicative of at least one of energy or force on two or more potential energy surfaces among potential energy surfaces of a molecular structure while using the molecular structure as input, and
the second state information is different from the two or more pieces of first state information output by the one or more inferencers, and indicates at least one of energy or force on the predetermined potential energy surface.

4. The information processing system according to claim 3, wherein
each of the one or more inferencers is a trained model that has been trained by machine learning, and the trained model has been trained to output the two or more pieces of first state information while using the molecular structure as input.

5. The information processing system according to claim 3, wherein
the outputter further outputs intersection information including at least one of energy at an intersection of the two or more potential energy surfaces, force at the intersection, or a molecular structure at the intersection.

6. The information processing system according to claim 5, wherein
in a case where the second state information and the intersection information each include a molecular structure, a molecular structure at an extreme value of the energy indicated by the second state information matches a molecular structure indicated by the intersection information.

7. The information processing system according to claim 3, wherein
the predetermined potential energy surface is a surface that is differentiable at least once.

8. The information processing system according to claim 3, wherein
the two or more potential energy surfaces are surfaces that use different spin multiplicities as parameters.

9. The information processing system according to claim 3, wherein
the molecular structure is a molecular structure that exhibits intersystem crossing, and
the one or more inferencers include a first inferencer that outputs first state information indicative of at least one of energy or force on a potential energy surface representing a singlet state of the molecular structure among the potential energy surfaces and a second inferencer that outputs first state information indicative of at least one of energy or force on a potential energy surface representing a triplet state of the molecular structure among the potential energy surfaces.

10. The information processing system according to claim 9, wherein
the second state information output by the outputter indicates the lower energy of the energy on the potential energy surface representing the singlet state and the energy on the potential energy surface representing the triplet state.

11. The information processing system according to claim 9, wherein
the second state information output by the outputter indicates the force on a potential energy surface with lower energy between the potential energy surface representing the singlet state and the potential energy surface representing the triplet state.

12. The information processing system according to claim 5, wherein
the searcher further includes a processor that performs processing at the intersection of the two or more pieces of first state information output by the one or more inferencers and generates the second state information by combining regions of potential energy surfaces where at least one of energy or force is lower than at least one of energy or force at the intersection in the two or more pieces of first state information.

13. An information processing method comprising:
causing a computer to derive two or more pieces of first state information indicative of at least one of energy or force on two or more potential energy surfaces among potential energy surfaces of a molecular structure including one or more molecules while using the molecular structure as input; and
causing the computer to output second state information indicative of at least one of energy or force on a predetermined potential energy surface different from the two or more potential energy surfaces on a basis of the two or more pieces of first state information.
